(19) United States
(12) Patent Application Publication
(10) Pub. No.: US 2025/0352715 A1
(43) Pub. Date: Nov. 20, 2025

(54) SYSTEMS AND METHODS FOR TRANSLUMINAL ENDOSCOPIC VACUUM

(71) Applicant: University of Washington, Seattle, WA (US)

(72) Inventors: Tom Mikolyuk, Seattle, WA (US); Anya Prasad, Seattle, WA (US); Srividhya Rajagopalan, Seattle, WA (US); Joseph Shim, Seattle, WA (US); Adam Templeton, Seattle, WA (US); Xiuqi Guo, Seattle, WA (US); Eric J. Seibel, Seattle, WA (US)

(21) Appl. No.: 19/208,444

(22) Filed: May 14, 2025

Related U.S. Application Data
(60) Provisional application No. 63/648,565, filed on May 16, 2024.

(51) Int. Cl. A61M 1/00

(52) U.S. Cl. CPC A61M 1/912; A61M 2205/3344

(57) ABSTRACT

Systems and methods for an endoscopic vacuum therapy system are herein provided. In one example, an endoscopic vacuum therapy system a negative pressure source; a tube comprising a plurality of openings at a distal end, the tube being fluidly coupled to the negative pressure source at a proximal end; and a fluid collection element coupled to the tube, wherein the fluid collection element is compressed via a segmented dissolvable casing.

### CROSS-REFERENCE TO RELATED APPLICATIONS

The present application claims priority to U.S. Provisional Application No. 63/648,565 entitled “TRANSLUMINAL ENDOSCOPIC VACUUM COMPONENTS AND METHODS OF USE”, and filed on May 16, 2024. The entire contents of the above-listed application are hereby incorporated by reference for all purposes.

### FIELD

Embodiments of the subject matter disclosed herein relate to systems, devices, and methods for a transluminal endoscopic vacuum.

### BACKGROUND

Wound vacuum-assisted closure (VAC) has been used in treatment of external skin wounds, such as acute surgical incisions and chronic wounds, to promote healing by applying negative pressure (e.g., vacuum suction) to the wound area. The wound VAC system removes fluid, purulent drainage, and debris via a sterile foam sponge. Due to the efficacy of wound VAC systems, the approach has been proposed for luminal injuries, such as fistulas and anastomotic leaks. This treatment, e.g., endoscopic vacuum therapy (EVT), may apply to transnasal, transoral, transrectal, and/or percutaneous approaches for lesions in the foregut (e.g., esophagus, stomach, and duodenum) or colon. However, EVT is currently performed using makeshift devices, such as suturing a sponge onto the end of a hollow tubed device that can facilitate suction, (Ex,. nasogastric tube (NG tube). Such makeshift devices are not standardized and can vary by application, approach, and clinician.

### BRIEF DESCRIPTION

In one example, an endoscopic vacuum therapy system is herein disclosed, wherein the endoscopic vacuum therapy system comprises a negative pressure source; a tube comprising a plurality of openings at a distal end, the tube being fluidly coupled to the negative pressure source at a proximal end; and a fluid collection element coupled to the tube, wherein the fluid collection element is compressed via a segmented dissolvable casing. In a first embodiment, the fluid collection element is directly coupled to the tube via an adhesive. In a second embodiment, the fluid collection element is coupled to the tube via an attachment system comprising an attachment clip and/or an attachment sheath.

Further, a third embodiment is herein disclosed, wherein the endoscopic vacuum therapy system comprises both a negative pressure source and a positive pressure source, wherein the tube is a double lumen tube and the fluid collection element comprises an inflatable vacuum device including a positive gauge pressure channel and a negative gauge pressure channel.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 shows an endoscopic vacuum therapy (EVT) system including a vacuum tube system;

FIG. 2 shows a detailed view of a proximal end of a tube of the vacuum tube system;

FIG. 3 shows a detailed view of a distal end of the tube of the vacuum tube system;

FIG. 4 shows the vacuum tube system according to a first embodiment of the present disclosure with a sponge in an expanded state;

FIG. 5 shows the vacuum tube system according to the first embodiment of the present disclosure with the sponge in a compressed state via a segmented dissolvable casing;

FIG. 6 shows a detailed view the proximal end of the vacuum tube system according to the first embodiment of the present disclosure with the sponge in the compressed state via the segmented dissolvable casing;

FIG. 7 shows a detailed view of a segment of the segmented dissolvable casing;

FIG. 8A shows a detailed view of the proximal end of the vacuum tube system comprising an attachment system according to a second embodiment of the present disclosure;

FIG. 8B shows the attachment system decoupled from the vacuum tube system, the attachment system including an attachment clip and an attachment sheath;

FIG. 9 shows a detailed view of the attachment clip of the attachment system;

FIG. 10 shows a detailed view of the attachment sheath of the attachment system;

FIG. 11 shows the vacuum tube system including a double lumen tube and an inflatable vacuum device according to a third embodiment of the present disclosure;

FIG. 12 shows the inflatable vacuum device;

FIG. 13 shows a partially cross-sectional view of the inflatable vacuum device;

FIG. 14 shows a flowchart illustrating a method for the EVT system according to the first embodiment;

FIG. 15 shows a flowchart illustrating a method for the EVT system according to the second embodiment;

FIG. 16 shows a flowchart illustrating a method for the EVT system according to the third embodiment;

FIGS. 17A-C show a second example of the inflatable vacuum device of FIG. 11; and

FIGS. 18A-C show a third example of the inflatable vacuum device of FIG. 11.

### DETAILED DESCRIPTION

The following description relates to various embodiments of a transluminal endoscopic vacuum therapy (EVT) system. More particularly, one or more embodiments of systems and methods for transluminal endoscopic vacuum for treatment of internal (e.g., luminal) wound sites resultant from fistulas, leaks, dehiscence, and the like are shown and described herein.

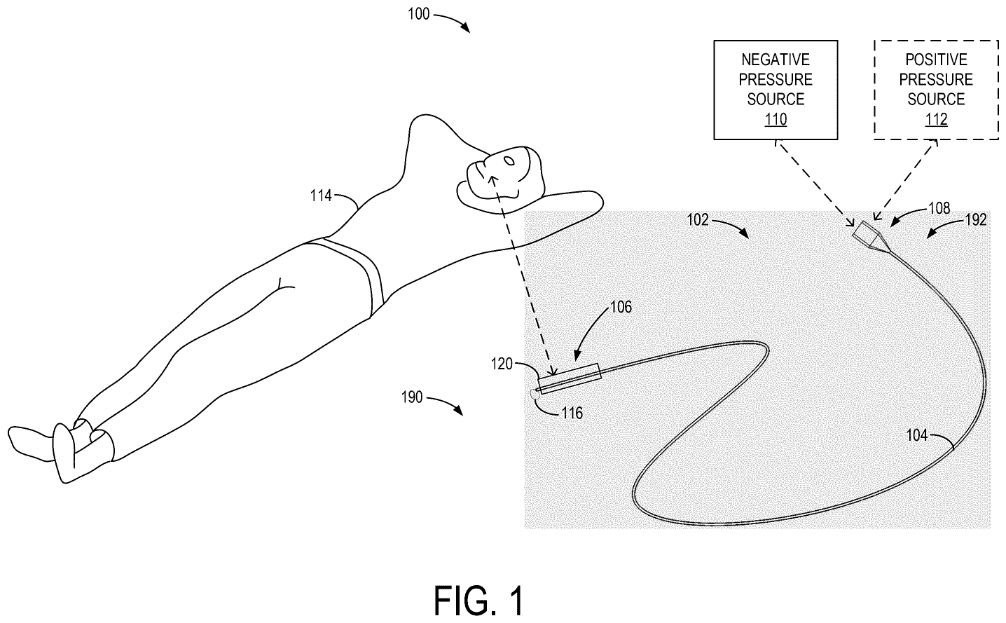

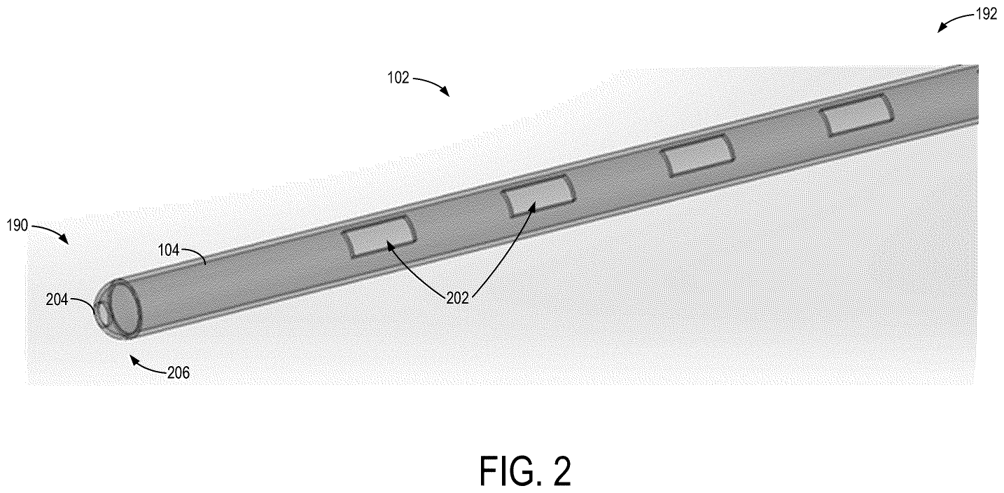

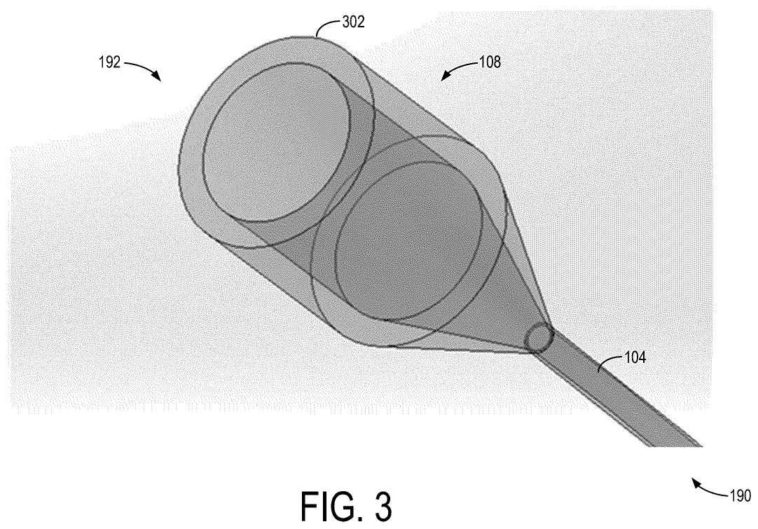

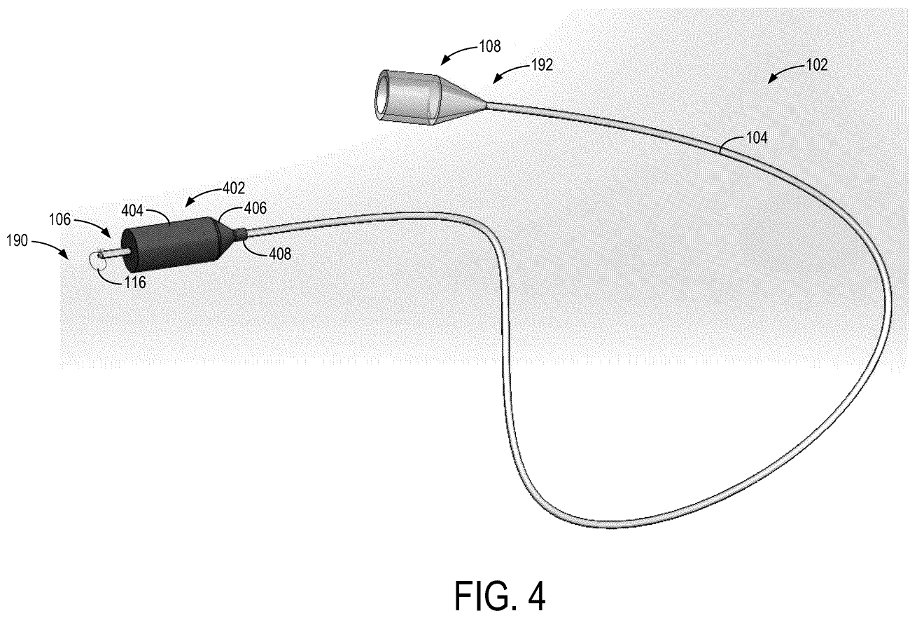

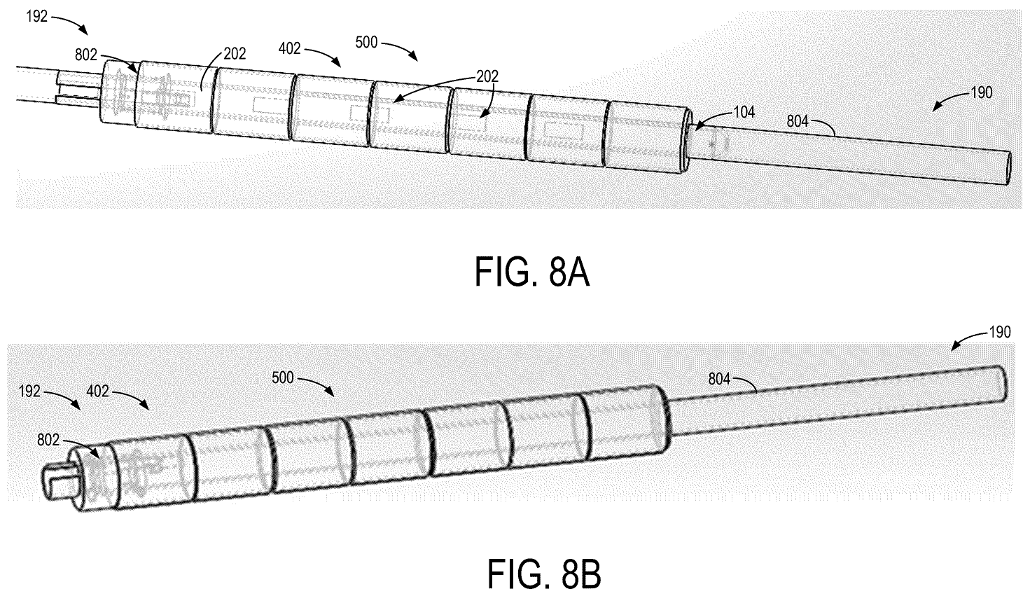

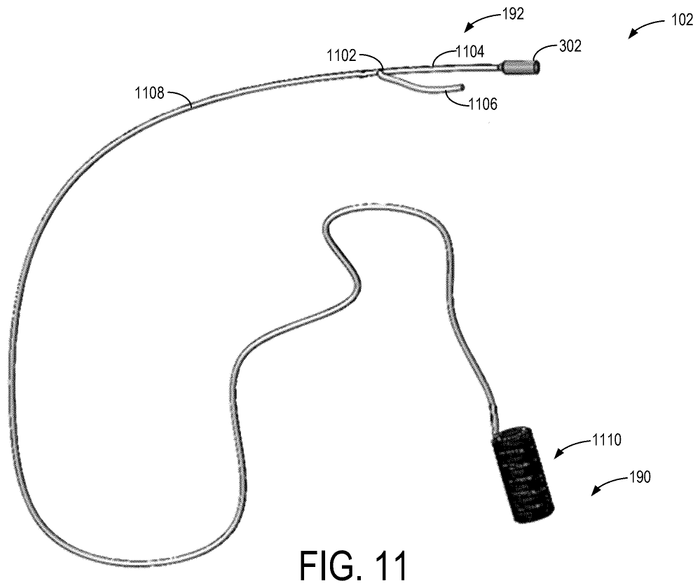

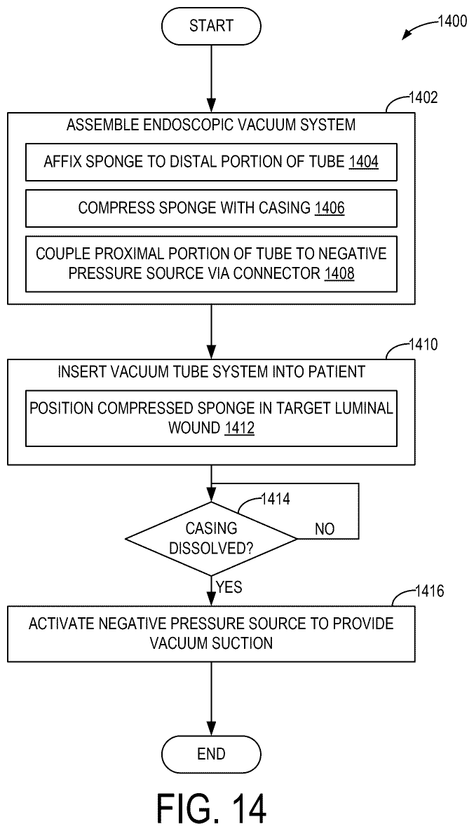

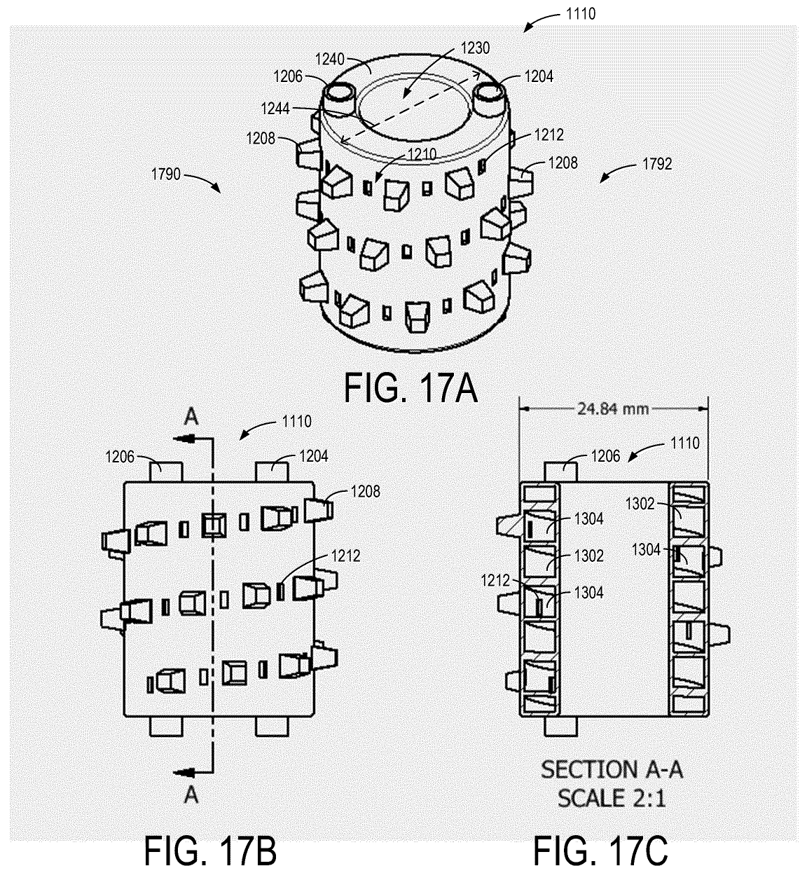

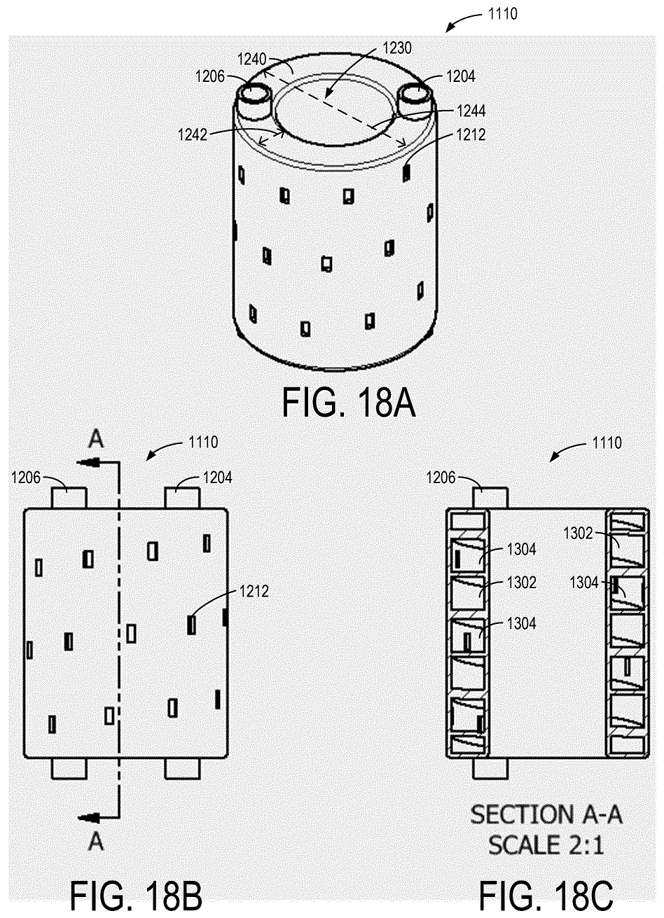

FIG. 1 shows a transluminal EVT system, including a vacuum tube system and a negative gauge pressure source. The vacuum tube system as herein described comprises a tube and a fluid collection element. FIGS. 2 and 3 show detailed views of the tube disassembled from the fluid collection element. FIGS. 4-7 show the vacuum tube system according to a first embodiment of the present disclosure. FIGS. 8A-10 show the vacuum tube system according to a second embodiment. FIGS. 11-13 show a third embodiment of the vacuum tube adapted for EVT. FIGS. 14-16 show flowcharts illustrating methods for one or more of the first, second, and third embodiments of the EVT system. FIGS. 17A-18C show alternative examples of the inflatable vacuum device according to the third embodiment.

Turning to FIG. 1, an exemplary EVT system 100 is shown. The EVT system 100 may comprise a vacuum tube system 102. The vacuum tube system 102 may be adapted for EVT according to one or more of the embodiments herein presented. For example, the vacuum tube system 102 may comprise tube 104 coupled to a fluid collection element 120 comprising luminal contacting material, such as an open-cell foam (e.g., in the form of a foam sponge) or an inflatable vacuum device arranged at a distal portion 106 towards a distal end 190 of the tube 104. A proximal portion 108 of the tube 104 may comprise a connector arranged at a proximal end 192. The distal portion 106 may be an insertion tip that is configured for a desired application, as will be further described below. In this context, the distal end 190 may be the end of the tube 104 that is to be inserted into a patient 114. For example, the vacuum tube system 102 may be similar to a modified nasogastric (NG) tube and the distal end 190 may be inserted transnasally (e.g., through the patient's nasal passage). The vacuum tube 102 may be adapted for other approaches as well, such as transrectal insertion, transoral insertion, or percutaneous insertion.

The tube 104 may be a flexible tube, for example formed of a polyvinyl chloride (PVC), silicone, or polyurethane (PU). “Flexible” is used in this context to indicate that the tube can be bent, angled, and positioned in various conformations, thus allowing the tube to be positioned for entry into a bodily orifice and maneuvered within the body to a desired position and orientation. The tube 104 may be single lumen or double lumen. For example, the tube 104 may comprise a single lumen when the EVT system 100 is configured according to a first embodiment, wherein the fluid collection element 120 is a foam sponge material and is directly coupled to the distal portion 106 of the tube 104, or according to a second embodiment, wherein the fluid collection element 120 is a foam sponge material and is coupled to the distal portion 106 via an attachment system. When the EVT system 100 is configured according to a third embodiment, wherein the fluid collection element 120 is an inflatable vacuum device coupled to the distal portion 106, the tube 104 may comprise a double lumen, for example comprising a lumen for negative pressure and a lumen for positive pressure. The double lumen may also provide a lumen for negative pressure and a lumen for instillation of fluids for nutrition or hydration.

The proximal portion 108 may be coupled to a negative gauge pressure source 110 and, in some embodiments, to a positive gauge pressure source 112. For example, in the first and second embodiments, the EVT system 100 may be configured for vacuum suction and thus the vacuum tube system 102 may be coupled to the negative pressure source 110. The negative pressure source 110 may be configured to generate and maintain a vacuum at a predetermined pressure. The negative pressure source 110 may thus comprise a pressure regulator, a pressure sensor, and the like for generating and maintaining the vacuum. The negative pressure source 110 may additionally comprise or be coupled to a secretion container that is configured to hold vacuumed material (e.g., serous fluids, sanguineous fluids, serosanguineous fluids, purulent drainage, bacterial and/or fungal debris, necrotic tissues, interstitial fluids, and/or residual surgical fluids like irrigation/saline).

The positive pressure source 112, when included such as in the third embodiment of the present disclosure, may be configured to provide positive pressure at a predetermined pressure. As will be further described below with respect to FIGS. 11-13, the positive pressure may be provided to the fluid collection element to maintain a diameter of an orifice. For example, the inflatable vacuum device may be positioned at anastomosis and positive pressure may be applied, such as to reduce potential anastomotic stricture. Negative pressure (e.g., vacuum) may be applied at the same site for wound vacuum assisted tissue healing and closure.

In some examples, the distal portion 106 of the tube 104 additionally comprises a suture loop 116 or other similar graspable component that is coupled to an atraumatic tip of the tube 104. The suture loop 116 may be utilized in positioning the vacuum tube system 102. For example, the suture loop 116 may be graspable, such as by grasping forceps affixed to or part of an endoscope, during placement and/or after deployment for adjustment of the positioning.

The distal portion 106 of the tube 104 is shown in greater detail in FIG. 2 without said suture loop. The example of the tube 104 shown in FIG. 2 comprises a single lumen and thus corresponds to the first and second embodiments described above and below. The distal portion 106 of the tube 104 may comprise a plurality of openings 202. The plurality of openings 202 may be openings (e.g., fenestrations) in the side wall of the tube 104 that expose the inner lumen to an external environment. In some examples, the plurality of openings 202 may be distributed circumferentially about the distal portion 106 of the tube. For example, the distal portion 106 may comprise one or more sets of holes each arranged in a line along a longitudinal axis of the tube 104. As a non-limiting example, the distal portion 106 may comprise two sets of five openings, the two sets being arranged at opposite sides (e.g., at a relative front and relative back) of the tube. For each set of openings, the openings may be spaced apart equidistant from each other. In another example, the distal portion 106 may comprise a single set of openings.

The distal portion 106 of the tube 104 may additionally comprise a hole 204 at a tip 206. The tip 206 may be rounded or tapered to facilitate insertion of the vacuum tube system 102. The hole 204 may be a circular or oval hole at a center of the tip 206. The hole 204 may be configured to allow a guide wire to pass through the lumen of the tube 104. For example, a guide wire may be inserted into the proximal end 192 of the tube 104 and fed through the lumen, exiting through the hole 204. In this way, a guide wire may be utilized with the EVT system 100 to aide in placement and positioning.

The proximal portion 108 of the tube 104, according to the first and/or second embodiments, is shown in greater detail in FIG. 3. The proximal portion 108 may comprise a connector 302. The connector 302 may be configured to mate with a port of the negative pressure source 110. The connector may comprise a Luer lock or slip connector, a funnel connector, a stepped connector, a straight tubing connector, or other type of connector. The type of connector may be configured based on the negative pressure source 110. In the example show in FIG. 3, the connector 302 is a cylindrical pressure fitting with a funnel tube connector.

Turning to FIG. 4, the vacuum tube system 102 is shown according to the first embodiment herein disclosed. In the first embodiment, the fluid collection element may comprise a sponge 402. The sponge 402 may comprise a foam material, such as black PU foam, white polyvinyl alcohol (PVA) foam, silver-impregnated foam, or similar. Other materials are also possible, such as gauze, hydrocolloids, non-woven polyester with silicone elastomer, and/or silver-impregnated sponges. Suction from the negative pressure source 110 may be provided through the sponge 402 when the sponge is arranged around the plurality of openings 202. The sponge 402 may be configured with an at least partially cylindrical shape with a central through opening (e.g., a hollow center).

The sponge 402, as will be further described below, may be configured to be compressed. The sponge 402 is shown in FIG. 4 in an expanded, neutral state. The sponge 402 may be configured to be compressed into a compressed state, wherein in the compressed state, a diameter of the sponge 402 is smaller than when the sponge 402 is in the expanded, neutral state. For example, the sponge 402 may be configured to be compressed via a dissolvable casing. The sponge 402 may be configured to be compressed to aid in entry through a bodily orifice. For example, a nasal passage of a patient may be smaller in diameter than the foam in its expanded state. For example, in the expanded state, the diameter of the sponge 402 may be 15-25 mm and in the compressed state, the diameter of the sponge 402 may be 4.5 to 5.5 mm. Thus, the sponge may be configured to be compressed so as to allow the sponge, in the compressed state with a smaller diameter, to enter through the nasal passage. Further, a target site for the sponge (e.g., a luminal wound) may have a diameter that is smaller than the sponge 402 in its expanded state, which may allow the sponge, when expanded back to its expanded state following dissolution of the dissolvable casing, to form a seal at the target site. Thus, the sponge being configured to compress may allow for navigation of the sponge to the target site.

The sponge 402 may comprise a main body 404 and a conical tip 406. The main body 404 may be configured as a cylinder shape. The conical tip 406 may be formed with or coupled to the main body 404. The conical tip 406 may further comprise a cylindrical extension 408. The cylindrical extension 408 may have a smaller diameter than the main body 404 when the main body 404 is in an expanded state. The main body 404 may be positioned over and/or in contact with the plurality of openings 202 of the tube 104. For example, the tube 104 may be inserted through the hollow center of the sponge 402 with the plurality of openings 202 arranged within the hollow center. In some examples, the conical tip 406 may be positioned proximal to the plurality of openings 202 and the cylindrical extension 408 may be positioned proximal to the rest of the conical tip 406.

The sponge 402 may be at least partially adhered to the distal portion 106. For example, the conical tip 406 may be adhered to the distal portion 106. In some examples, only the cylindrical extension 408 of the conical tip 406 may be adhered to the tube 104, for example proximal to a most proximal opening of the plurality of openings 202. Portions of the sponge 402 that are positioned over or in contact with the plurality of openings 202 may not be adhered to the tube 104 in order to avoid plugging the openings with the adhering material (e.g., silicone adhesive, medical grade epoxy, etc.). Thus, suction may be provided through the sponge 402 in order to supply vacuum therapy to a target area (e.g., to a luminal wound). The conical tip 406 of the sponge 402 may be permanently, temporarily, or selectively affixed to the tube 104. As a non-limiting example, the conical tip 406 may be affixed to the tube 104 using an adhesive (e.g., silicone adhesive, medical grade epoxy, etc.) that does not dissolve when in contact with bodily fluids but does dissolve when exposed to an adhesive dissolving agent like acetone or isopropyl alcohol (e.g., for detachment of the sponge after removal of the vacuum system). For example, adhesion may be done using a biocompatible adhesive such as Permabond.

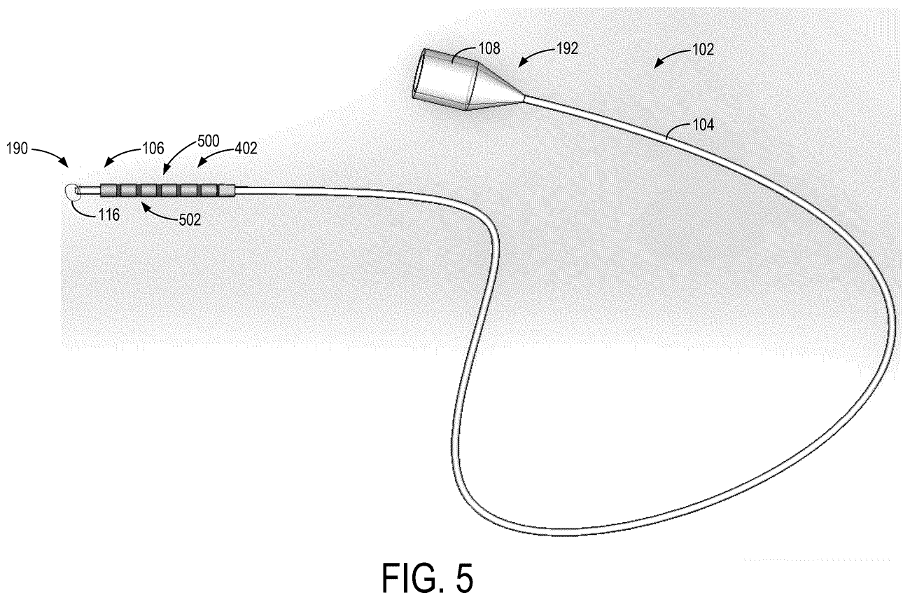

FIG. 5 shows the vacuum tube system 102 according to the first embodiment of the present disclosure with the sponge 402 in the compressed state. In some examples, the sponge, adhered to the tube as described above, may be fed through a funnel with a diameter smaller than the diameter of the sponge in the compressed state (e.g., 4-5 mm). Feeding the sponge through the funnel may compress the sponge further than the compressed state of the sponge to allow a dissolvable casing 500 to be placed about the sponge 402. The vacuum tube system 102 may be inserted into an anatomical or fabricated orifice of the patient 114. For example, the vacuum tube system 102 may be inserted transnasally for treatment of esophageal, gastric, or duodenal luminal wounds (e.g., fistulas, anastomoses, anastomotic leaks, etc.). During insertion, the sponge 402 may be compressed for ease of insertion. The sponge 402 may be temporarily maintained in the compressed state via the dissolvable casing 500. The dissolvable casing 500 may comprise a plurality of gel shells 502. As will be described further below, the dissolvable casing 500 may be a segmented dissolvable casing, wherein each of the plurality of gel shells 502 is a segment of the segmented dissolvable casing.

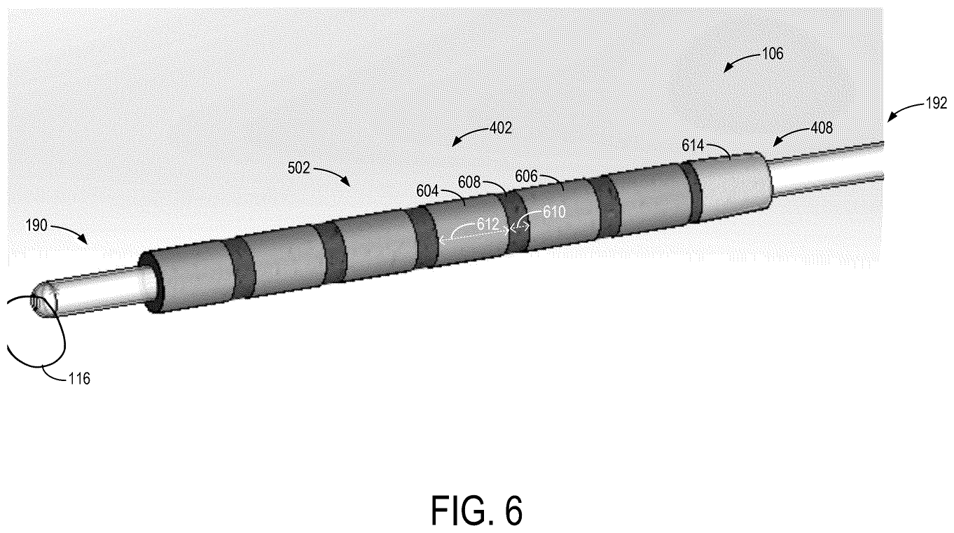

FIG. 6 shows the sponge 402 in the compressed state in greater detail. As noted, the sponge 402 may be compressed via a segmented dissolvable casing 500 formed of the plurality of gel shells 502. Each of the plurality of gel shells 502 may be positioned circumferentially about the compressed sponge 402. The plurality of gel shells 502 may be spaced apart from one another, thereby providing flexibility to the distal portion 106, which may increase maneuverability of the tube during positioning. As an example, a first gel shell 604 may be separated from an adjacent second gel shell 606 by a gap 608. The gap 608 may have a first width 610 and the first and/or second gel shells 604, 606 may have a second width 612. In the example shown, the first width 610 is less than the second width 612. In other examples, the first width 610 may be greater than the second width 612. As non-limiting examples, the first width 610 may be between 0.5 and 3 mm and the second width 612 may be between 3 and 10 mm.

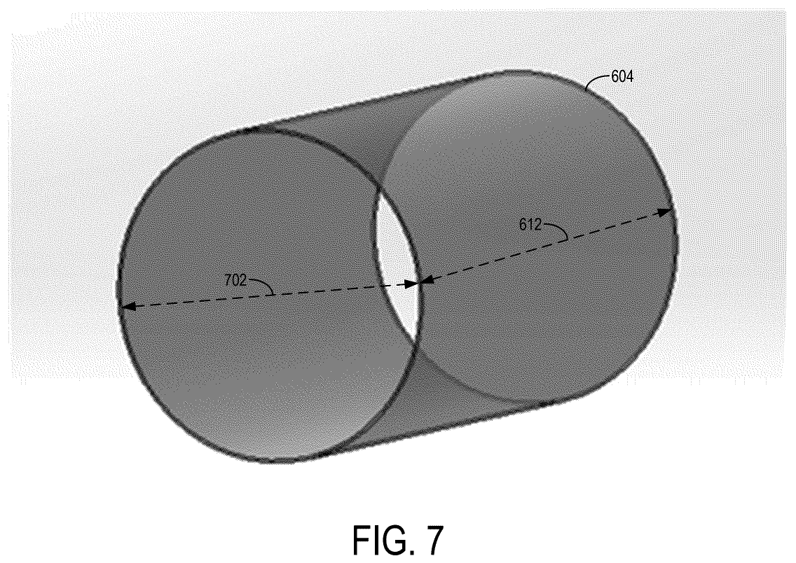

Turning briefly to FIG. 7, the first gel shell 604 is shown disassembled from the vacuum tube system 102, the first gel shell 604 being an example of one of the plurality of gel shells 502 (e.g., a segment of the segmented dissolvable casing 500). As shown in FIG. 7, an inner diameter 702 of one or more of the gel shells 502 may be equal to or slightly greater than an outer diameter of the compressed sponge 402. For example, the inner diameter 702 may be between 4.9-5.5 mm.

Returning to FIG. 6, a proximal gel shell 614 may be arranged circumferentially about the cylindrical extension 408. In some examples, the cylindrical extension 408 may be tapered, narrower in cross-section towards the proximal end 192 and wider in cross-section towards the distal end 190. The proximal gel shell 614 may likewise be tapered to be narrower towards the proximal end 192 compared to the distal end 190. In some examples, a gap between the proximal gel shell 614 and a next distally adjacent gel shell may be smaller than the other gaps (e.g., gap 608). In other examples, the gap between the proximal gel shell 614 and the next distally adjacent gel shell may be greater than or equal in size to the other gaps (e.g., gap 608).

The plurality of gel shells 502 may be made of animal-derived gelatin or a vegetarian alternative material (e.g., hydroxypropyl methylcellulose (HPMC)). The plurality of gel shells 502 may be configured to absorb moisture and/or environmental compounds, for example from gastric fluids (e.g., including Pepsin, a digestive enzyme in gastric fluid). Absorbing this moisture may result in the gel shells swelling, softening, and ultimately dissolve. As the gel shells dissolve, the sponge 402 may expand back to its neutral, uncompressed state.

A thickness of the gel shells may determine how quickly the gel shells dissolve. In some examples, each of the plurality of gel shells 502 may be the same thickness, thus resulting in relatively simultaneous dissolution. In other examples, the thicknesses of the gel shells 502 may vary. For example, more proximal gel shells may have a smaller thickness than more distal gel shells, thus resulting in varied dissolution where a distal portion of the sponge 402 expands after the proximal portion. As an example, the distal portion of the sponge 402 may be positioned at a distal side of a luminal wound when the sponge 402 is compressed, whereby the distal portion enters through the luminal wound and then resides in the desired position at the distal side. Thus, the gel shells at the distal portion may dissolve later than the proximal gel shells, allowing more time for positioning of the distal portion. Further, a thickness of each of the plurality of gel shells 502 may depend on the particular application. For example, the gel shells may be thicker for colonic applications compared to gastric applications.

In a non-limiting example, the thickness of the gel shells may be configured for dissolution after 2-5 minutes. This timeframe may allow a clinician to appropriately position the vacuum tube system 102 as desired before dissolution and resultant expansion of the sponge 402.

FIG. 14 shows a flowchart illustrating a method 1400 for the EVT system 100 according to the first embodiment described above. The method 1400 is herein described with reference to the components of the system 100 with regard to the first embodiment of the present disclosure, wherein the system comprises a negative pressure source (e.g., negative pressure source 110) and a vacuum tube system (e.g., vacuum tube system 102) comprising a tube (e.g., tube 104), a connector (e.g., connector 302), and a fluid collection element (e.g., sponge 402).

At 1402, method 1400 includes assembling the EVT system. Assembling the EVT system may comprise assembling the vacuum tube system by affixing the sponge to the distal portion of the tube, as noted at 1404. Affixing the sponge to the distal portion of the tube may comprise adhering (e.g., gluing) a conical tip and/or a cylindrical extension portion of the sponge to an external surface of the tube. Assembling the vacuum tube system may additionally comprise compressing the sponge with a dissolvable casing positioned around the compressed sponge, as noted at 1406. As described above, the dissolvable casing may comprise a segmented casing formed of a plurality of gel shells. Assembling the EVT system may further comprise coupling a proximal portion of the tube to the negative pressure source via the connector, as noted at 1408.

At 1410, method 1400 includes inserting the vacuum tube system into a patient. In one example, the vacuum tube system may be inserted transnasally. Such may be the case for upper gastrointestinal luminal wounds, such as esophageal, gastric, or duodenal fistulas or perforations, anastomotic leaks, or the like. In another example, the vacuum tube system may be inserted transrectally, for example in the case of lower gastrointestinal luminal wounds, such as colonic fistulas, colonic perforations, anastomotic leaks, or the like. Other insertion approaches are possible, such as transorally and/or percutaneously. During insertion, the compressed sponge may be positioned at a target site, such as in a target luminal wound, as noted at 1412. For example, the compressed sponge may be positioned to span the luminal wound, with a distal portion of the sponge being positioned at a distal side of the wound and a proximal portion of the sponge being positioned at a proximal side of the wound. In some examples, a graspable component, such as a suture loop (e.g., suture loop 116) may be used to aid in positioning the vacuum tube system at the target site. For example, grasping forceps mounted at a distal end of an endoscope may be used to grab the suture loop, allowing the clinician to position the compressed sponge at the target site more accurately.

At 1414, method 1400 includes determining if the dissolvable casing has dissolved. In some examples, a predetermined amount of time may be set, after which the gel shells of the casing may be considered dissolved, based on the general or average dissolution time of the gel shells for the given thickness(es). In other examples, gel shell dissolution may be visually assessed, for example via an endoscope or an imaging guide wire that is inserted with the tube system (e.g., through lumen and tip hole 204). If the casing has not dissolved, method 1400 repeats 1414 until the casing has dissolved (e.g., waits). Once the casing has dissolved, method 1400 proceeds to 1416.

At 1416, method 1400 includes activating the negative pressure source to provide vacuum suction. As described above, the gel shells may dissolve after a period of time or varying periods of time and in response, the sponge may expand to its neutral, uncompressed state. The tube, as described above, may comprise a plurality of openings (e.g., fenestrations). The negative pressure source may create vacuum suction, which is distributed through the sponge that is placed inside the wound. The sponge may allow for an even application of the suction across the entire wound surface. With the negative pressure source activated, the vacuum suction may pull wound exudate, blood, infectious material, and other fluid secretions through the sponge and through the lumen of the tube, to be collected in a secretion container of or coupled to the negative pressure source.

In some examples, the pressure from the negative pressure source may be varied over time after activation. For example, as a wound at the target site heals, and thus closes or otherwise reduces in size, the vacuum suction may be modified during operation of the system. For example, less suction power may be demanded due to fluid leakage rates declining during healing or more suction power may be demanded to overcome loss of porosity of the sponge due to clogging of pores in the foam during the healing process.

In some examples, one or more components of the EVT system according to the first embodiment may be reusable. For example, in some examples, the tube of the vacuum tube system and the negative pressure source may be reusable, wherein the tube can be sterilized for repeat usages. In some examples, one or more components of the EVT system according to the first embodiment may be disposable. For example, the sponge may be disposable.

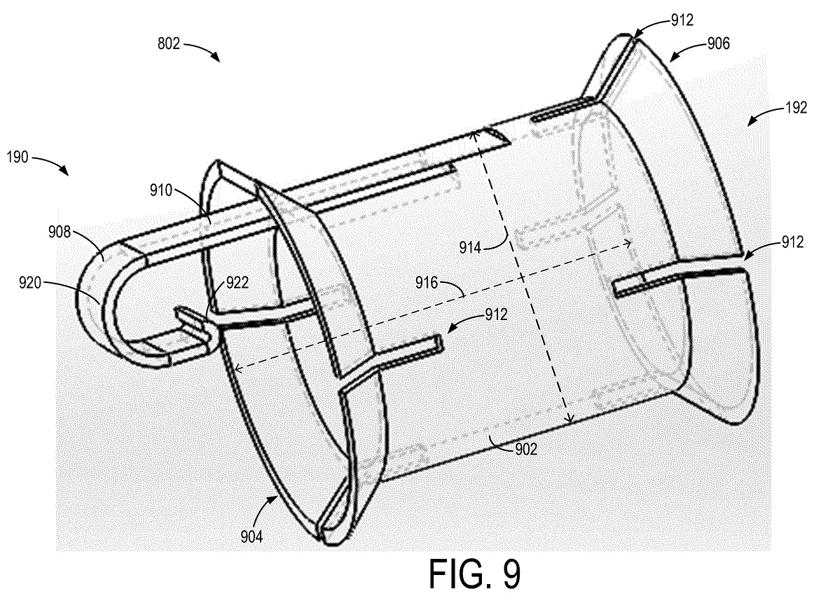

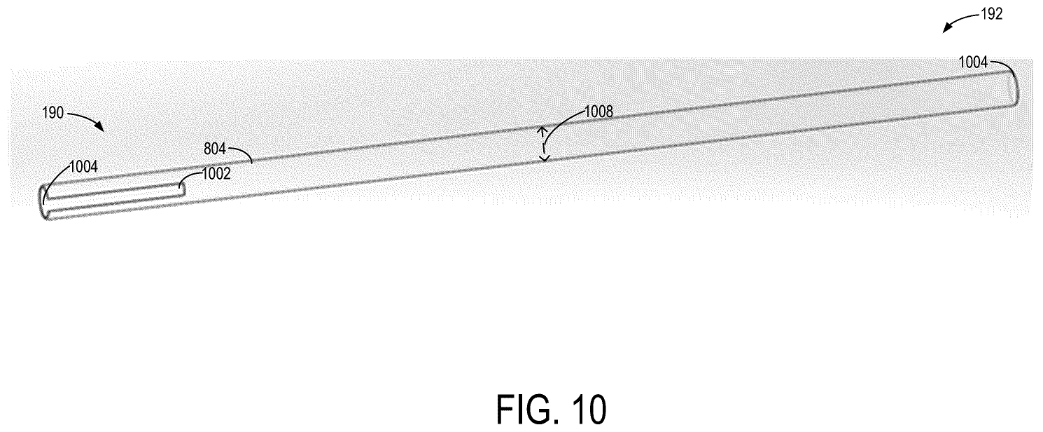

Turning now to FIGS. 8A-10, the vacuum tube system 102 according to the second embodiment is shown herein. In the second embodiment, the fluid collection element 120 may comprise the sponge 402 fixedly coupled to an attachment clip 802 of an attachment system. The sponge 402 may be coupled to the tube 104 via this attachment system, including the attachment clip 802 and an attachment sheath 804. FIG. 8A shows the sponge 402 assembled with the tube 104 and FIG. 8B shows the sponge 402 disassembled from the tube 104. The attachment clip 802 is shown in greater detail in FIG. 9 and the attachment sheath 804 is shown in greater detail in FIG. 10. In the second embodiment, in some examples, the sponge 402 may be configured as a cylinder (e.g., without the conical tip). In other examples however, the sponge 402 may include the conical tip in the second embodiment, similar to as described above for the first embodiment.

The attachment clip 802 may be inserted into a proximal portion of the sponge 402 (e.g., towards the proximal end 192 of the sponge 402). The dissolvable casing 500, as described above, may be arranged circumferentially about the sponge 402. In other examples, the dissolvable casing 500 may be partially circumferential about the sponge 402. For example, the dissolvable casing 500 may encompass 330°, 300°, 270°, 240°, or other degree less than 360° about the sponge 402. The attachment sheath 804 may be positioned within the attachment clip 802. For example, an outer surface of the attachment sheath 804 may contact an inner surface of the attachment clip 802.

FIG. 9 shows the attachment clip 802 in greater detail. The attachment clip 802 may comprise a hollow cylindrical main body 902, a distal flange 904, a proximal flange 906, and a hook 908. In some examples, the hollow cylindrical main body 902, the distal flange, 904, and the proximal flange 906 may be formed as a single piece. The distal and proximal flanges 904, 906 may each comprise one or more notches 912. The one or more notches 912 may increase the flexibility and/or bendability, thereby allowing the flanges to be bent or flexed to match the demands of the sponge 402 and/or the application. In a non-limiting example, the flanges may be 0.5 to 3 mm in length with between a 15 and 60-degree slope. As a non-limiting example, the flanges may be 1 mm in length with an approximately 30-degree slope. Thus, the flanges may have a greater diameter than the hollow cylindrical main body 902, for example by a margin of between 5-10 mm. The attachment clip 802 may have an internal diameter 914 and may be a length 916 (excluding the hook 908). The length 916 may be 7-8 mm, in a non-limiting example.

The hook 908 may be arranged towards the distal end 190. The hook 908 may be coupled to the hollow cylindrical main body 902 via a connector 910. The hook 908 may be shaped and sized to mate with one of the plurality of openings 202. For example, each of the plurality of openings 202 may be approximately 2 mm across and the hook 908 may be approximately 1.5 mm across, thereby allowing the hook 908 to fit within one of the openings 202.

The hook 908 may comprise a major hook 920 and a minor hook 922. The major hook 920 may have a larger radius of curvature than the minor hook 922. As a non-limiting example, the major hook 920 may have a radius of curvature of approximately 1 mm and the minor hook 922 may have a radius of curvature of approximately 0.5 mm. The major hook 920 may be concave facing the proximal end 192 and the minor hook 922 may be concave facing the distal end 190. The minor hook 922 may extend from an end (e.g., a proximal end) of the major hook 920, the minor hook 922 then hooking back towards the major hook 920. The minor hook 922 may reduce shearing and tearing of the tube 104 by the major hook 920 during installation of the tube 104.

The hook 908 may be formed of a rigid material to decrease the possibility of the hook backing out of a tube to which it is connected. For example, the hook 908 may be a metal material, such as stainless steel, titanium, or the like. The hook 908, when assembled with the sponge 402, may be entirely covered by the sponge 402 to reduce contact between the metal hook and the patient's internal tissues.

FIG. 10 shows the attachment sheath 804 in greater detail. The attachment sheath 804 may be configured as a hollow cylinder with a notch 1002 at one end, in practice this may be the proximal end 192. The attachment sheath 804 may be configured as a hollow cylinder, wherein the attachment sheath 804 may include opening 1004 that extends through the entire sheath. A diameter 1008 of the attachment sheath 804 may be configured to fit within the internal diameter 914 of the attachment clip 802.

When the attachment sheath 804 is assembled with the fluid collection element (e.g., comprising the sponge affixed to the attachment clip), the notch 1002 may be aligned with the hook 908. In this way, the attachment sheath 804 may be positioned to at least partially extend past the clip (e.g., more towards the proximal end). The attachment sheath 804 may be more rigid than the sponge 402 and the tube 104. In this way, the attachment sheath 804 may increase the ease of installation by providing a rigid, smooth surface for the tube 104 to slide through, thereby reducing friction and flexion that would otherwise occur between the tube 104 and the sponge 402. The smooth surface of the attachment sheath 804 may give it the ability to slide out of the foam material of the sponge easily following installation of the tube 104. While not shown in the figures, the attachment sheath 804 in some examples may comprise a handle to aid in gripping the sheath during insertion and removal. In some examples, the attachment sheath 804 may be made of the same material as the attachment clip 802, such as stainless steel.

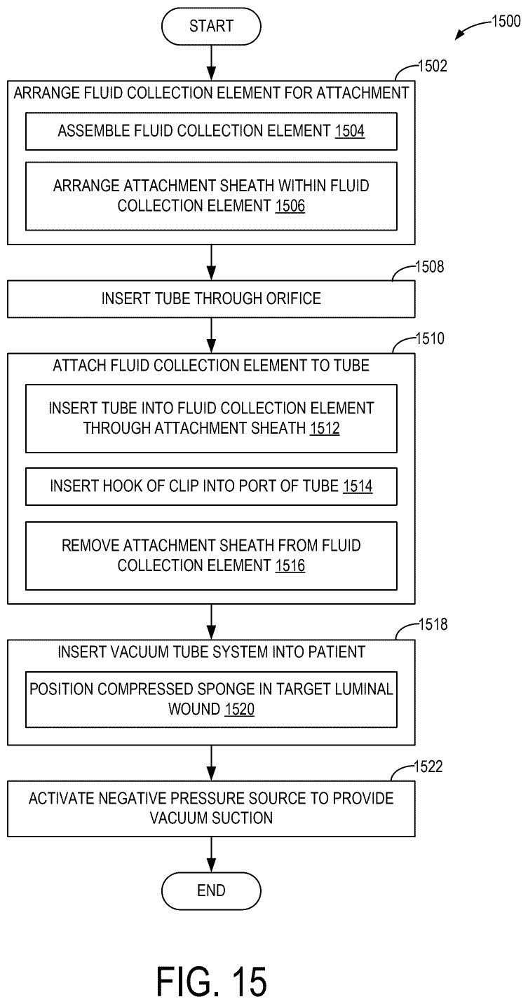

FIG. 15 shows a flowchart illustrating a method 1500 for the EVT system according to the second embodiment described herein. The method 1500 is herein described with reference to the components of the system 100 with regard to the second embodiment of the present disclosure, wherein the system comprises a negative pressure source (e.g., negative pressure source 110) and a vacuum tube system (e.g., vacuum tube system 102) comprising a tube (e.g., tube 104), a connector (e.g., connector 302), a fluid collection element (e.g., sponge 402), and an attachment system, such as described with respect to FIGS. 8A-10.

At 1502, method 1500 includes arranging the fluid collection element for attachment. As described above, the fluid collection element may comprise the foam sponge coupled to an attachment clip of the attachment system. Arranging the fluid collection element for attachment may comprise assembling the fluid collection element, as noted at 1504. Assembling the fluid collection element may comprise arranging the attachment clip within an internal cavity of the sponge at a proximal end (e.g., the end that will be positioned more towards the negative pressure source). After the clip is installed within the sponge, the proximal end of the sponge may be compressed and infused with epoxy. In some examples, the sponge may be coated with an adhesive material as well. The adhesive material may maintain a position of the clip as the epoxy is infused. The epoxy may change the texture of the proximal end of the sponge to a composite texture. Flanges of the attachment clip may serve as guards to maintain a position of the clip within sponge during insertion of the sponge into the patient, thereby providing additional hold between the sponge and the clip in addition to the epoxy and the adhesive.

Additionally, arranging the fluid collection element for attachment may comprise arranging the attachment sheath within the fluid collection element, as noted at 1506. As described above, the attachment sheath may be arranged within the fluid collection element, for example within the attachment clip and the sponge. A notch of the attachment sheath may be oriented to align with a hook of the clip, thus the attachment sheath may extend past the clip in the proximal direction by at least the length of the hook.

Similar to as described above, the sponge may be compressed, with a dissolvable casing being arranged circumferentially around the sponge to temporarily maintain the compressed state of the sponge. In some examples, the sponge may be compressed and the dissolvable casing positioned prior to placement of the clip and/or sheath. In other examples, the sponge may be compressed and the dissolvable casing positioned after placement of the clip and/or sheath.

At 1508, method 1500 includes inserting the tube of the vacuum tube system through an orifice of a patient. In one example, the orifice may be a nasal passage. The attachment system as herein described with respect to the second embodiment may be applicable when the sponge, in the compressed state, has too great an outer diameter for the orifice. For example, a target luminal wound may have a diameter that demands an expanded sponge diameter of a particular size. The sponge, when configured with this particularly sized expanded sponge diameter, may have a compressed diameter that is larger than a diameter of the patient's nasal passage. In such examples, the tube 104 of the vacuum tube system may be inserted through the patient's nasal passage first before attachment of the fluid collection element. Thus, in order to attach the fluid collection element to the tube, the tube may be inserted through the orifice and pulled out of another orifice. For example, the tube may be inserted through the nasal passage and then pulled out the mouth, for example by grasping a suture loop that is tied to a tip of the tube (e.g., suture loop 116).

At 1510, method 1500 includes attaching the fluid collection element to the tube of the vacuum tube system. Attaching the fluid collection element to the tube may comprise inserting the tube into the fluid collection element through the attachment sheath, as noted at 1512. During insertion of the tube into the fluid collection element, the hook of the clip may slightly compress an outer surface of the tube. The hook of the clip may then be inserted into one of a plurality of openings of the tube, as noted at 1514. As an example, the hook may be inserted into a proximal most opening of the plurality of openings. Once the hook is coupled to the given opening, the attachment sheath may be removed from the fluid collection element, as noted at 1516. As described above, the attachment sheath may be slid out of the sponge towards the distal end. Once the fluid collection element is attached to the tube and the sheath is removed, the vacuum tube system may be fully assembled.

In some examples, the tube may be inserted through the sheath with the openings aligning with the hook. The tube may be inserted such that a proximal most opening of the plurality of openings is positioned distal to the hook. The tube may then be pulled back (e.g., pulled towards the proximal end of the fluid collection element), allowing the hook to couple to the proximal most opening. In another example, the tube may be inserted into the fluid collection element with the plurality of openings oriented to not align with the hook. Then, once the plurality of openings are fully inserted into the sponge, e.g., distally past the clip, the tube may be twisted to align with the hook so that the opening into which the hook is to be inserted aligns with the hook. In this way, the hook may avoid prematurely catching on distally arranged opening. The hook may be arranged to maintain a relative position of the fluid collection element with respect to the tube, the major and minor hooks preventing the clip and/or sponge from backing out of the tube and decoupling from the tube when inserted inside the patient.

The fluid collection element may be attached to the tube while the tube is positioned through the orifice of the patient. As a non-limiting example, the tube may be inserted through the patient's nasal passage and then pulled out through the mouth. The fluid collection element may be attached to the tube via the attachment system herein described while the tube is held outside the patient's mouth (or held within the patient's open mouth). Then, the assembled vacuum tube may be positioned back within the patient's mouth to be inserted transorally.

At 1518, method 1500 includes inserting the vacuum tube system into a patient. As described above, the tube may be already positioned within the patient's mouth following attachment of the fluid collection element. The vacuum tube system (e.g., the tube+the fluid collection element) may then be inserted transorally. During insertion, the compressed sponge may be positioned in a target luminal wound, as noted at 1520. For example, the compressed sponge may be positioned to span the luminal wound, with a distal portion of the sponge being positioned at a distal side of the wound and a proximal portion of the sponge being positioned at a proximal side of the wound. In some examples, the suture loop may again be used to aid in positioning. For example, the suture loop may be grasped by forceps or other graspers during positioning at the target site.

At 1522, method 1500 includes activating the negative pressure source to provide vacuum suction. As described above, the gel shells may dissolve after a period of time or varying periods of time and in response, the sponge may expand to its neutral, uncompressed state. The tube, as described above, may comprise a plurality of openings. The negative pressure source may create vacuum suction, which is distributed through the sponge that is placed inside the wound. The sponge may allow for an even application of the suction across the entire wound surface. With the negative pressure source activated, the vacuum suction may pull wound exudate, blood, infectious material, and other fluid secretions through the sponge and through the lumen of the tube, to be collected in a secretion container of or coupled to the negative pressure source.

In some examples, the pressure from the negative pressure source may be varied over time after activation. For example, as a wound at the target site heals, and thus closes or otherwise reduces in size, the vacuum suction may be modified during operation of the system. For example, less suction power may be demanded due to fluid leakage rates declining during healing or more suction power may be demanded to overcome loss of porosity of the sponge due to clogging of pores in the foam during the healing process.

The second embodiment, including the attachment system herein described, provides an option for affixing the fluid collection element to the tube even when the patient's entry orifice is too small to allow passage of the compressed fluid collection element. The attachment sheath provides for smooth, quick entry of the tube into the sponge and the attachment clip, via its hook, quickly and easily affixes the sponge to the tube. In this way, the foam sponge may be quickly and easily attached to the tube after the tube has been inserted through the orifice (e.g., the nasal passage).

In some examples, one or more components of the EVT system according to the second embodiment may be reusable. For example, in some examples, the tube of the vacuum tube system and the negative pressure source may be reusable, wherein the tube can be sterilized for repeat usages. In some examples, one or more components of the EVT system according to the second embodiment may be disposable. For example, the sponge may disposable.

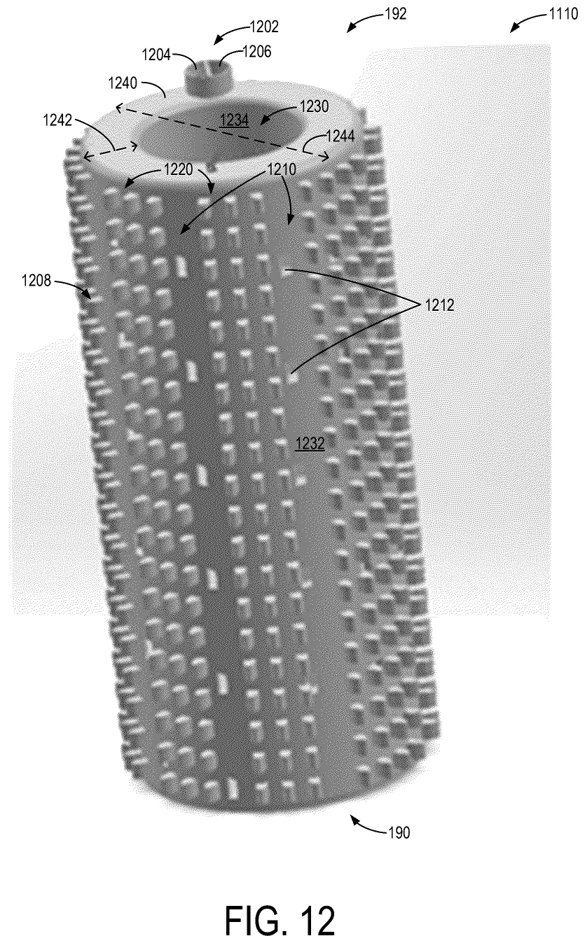

Turning now to FIGS. 11-13 and 17A-18C, the vacuum tube system 102 of the EVT system 100 is shown according to the third embodiment. FIG. 11 shows the vacuum tube system 102, wherein the fluid collection element 120 is an inflatable vacuum device 1110. In the third embodiment, the tube may include a double lumen tube 1108, a first lumen 1104 adapted for negative pressure and a second lumen 1106 adapted for positive pressure. The first lumen 1104 may be fluidly coupled to the negative pressure source 110, for example via connector 302. The second lumen 1106 may be fluidly coupled to the positive pressure source 112, for example via a second connector (not shown). In some examples, the first and second lumens 1104, 1106 may be separate tubes at the proximal end 192 for connection to the negative and positive pressure sources 110, 112, and then may join at a juncture 1102 to form the double lumen tube 1108. The inflatable vacuum device 1110 is shown in various examples in FIGS. 12-13, 17A-C, and 18A-C.

With positive pressure available, the vacuum tube system 102 may act both as a wound suction system as well as a stent system. As a non-limiting example, for an end-to-end anastomosis, the fluid collection element of the vacuum tube system may be positioned centered at the anastomosis to provide positive pressure as a stent, for example to provide the benefits of maintenance of luminal integrity allowing for passage of luminal secretions and/or nutrition to flow through the hollow lumen as well as anchoring in the situation of a tight anastomosis or luminal stricture or narrowing at the site of the leak. The negative pressure may act to promote healing of the anastomosis and treat, if present, anastomotic leakage. The healing process is expected to include a large variation of amounts of secretions, and changes in sizes and pressures from changes in tissue inflammation that can constrict the lumen. Thus, the device can be made responsive to these changes by varying the levels of negative and positive gauge pressures, creating a stent-like device with dynamic functionalities in size, shape, and suction.

In some examples, the double lumen tube 1108 may be configured with the first and second lumens 1104, 1106 arranged coaxially. For example, the first lumen 1104 may be arranged in a center of the second lumen 1106. The negative pressure lumen may be more rigid and in some examples thicker than the positive pressure lumen and thus, to maintain flexibility of the double lumen tube 1108, the first lumen 1104 may be arranged within the second lumen 1106. In other examples, the double lumen tube 1108 may be configured with the first and second lumens 1104, 1106 arranged adjacent to each other.

The inflatable vacuum device 1110 may be formed of one or more materials such as silicone, PU, polyethylene terephthalate (PET), and/or the like. The inflatable vacuum device 1110 may be at least partially expandable. For example, portions of the inflatable vacuum device 1110 that are in fluid communication with the second lumen 1106 may be inflated upon activation of the positive pressure source 112, providing outward pressure against the target patient anatomy (e.g., esophageal mucosa, colonic mucosa, etc.).

FIG. 12 shows the inflatable vacuum device 1110 in greater detail according to a first example. The inflatable vacuum device 1110 may be configured as a cylindrically shaped tube (e.g., a hollow cylinder). The inflatable vacuum device 1110 may thus comprise a hollow passage 1230 arranged within a body 1240. The body 1240 may have a thickness 1242. An outer wall 1232 may face outward, e.g., towards the patient's mucosa when inserted (e.g., colonic mucosa, esophageal mucosa, etc.) and an inner wall 1234 may define boundaries of the hollow passage 1230. When the inflatable vacuum device 1110 is positioned at a target anatomy, for example at a colonic anastomosis, the hollow passage 1230 may continue to allow passage of materials (e.g., human waste materials), thereby reducing potential for blockage or obstruction.

The inflatable vacuum device 1110 may comprise a lumen connecting port 1202 coupled to the body 1240. The lumen connecting port 1202 may comprise at least two lumen ports. For example, the lumen connecting port 1202 may comprise a first lumen connecting port 1204 and a second lumen connecting port 1206. In some examples, the first and second lumen connecting ports 1204, 1206 may be positioned adjacent to each other (e.g., via a shared central wall) as shown in FIG. 12 or positioned coaxially. In other examples, the first and second lumen connecting ports 1204, 1206 may be separated (e.g., on opposing sides of the body 1240), as in the examples shown in FIGS. 17A-18C described below.

The first lumen connecting port 1204 may be configured to fluidly couple to the first lumen 1104 and the second lumen connecting port 1206 may be configured to fluidly couple to the second lumen 1106. Thus, the lumen connecting port 1202 may be arranged in a configuration that matches the double lumen tube 1108. For example, when the double lumen tube 1108 is arranged with adjacent lumens, the lumen connecting port 1202 may be arranged with the two lumen ports adjacent to each other. Similarly, when the double lumen tube 1108 is arranged with coaxial lumens, the lumen connecting port 1202 may be arranged with coaxial lumen ports.

The inflatable vacuum device 1110 may additionally comprise, in some examples, a plurality of protrusions 1208 protruding away from the outer wall 1232. In the first example shown in FIG. 12, the plurality of protrusions may be arranged in columns that extend from the proximal end 192 to the distal end 190. In one example, the columns may be grouped together, for example in sets 1220, e.g., sets of three columns, wherein the sets 1220 are spaced apart by gaps 1210. One or more openings 1212 may be included in the outer wall 1232 of the inflatable vacuum device 1110, for example positioned at the gaps 1210. The one or more openings 1212, as will be further described below, may be positioned to correspond to a negative pressure channel within the inflatable vacuum device 1110. The one or more openings 1212 may therefore be similar to the plurality of openings 202 described above with respect to the first and second embodiments.

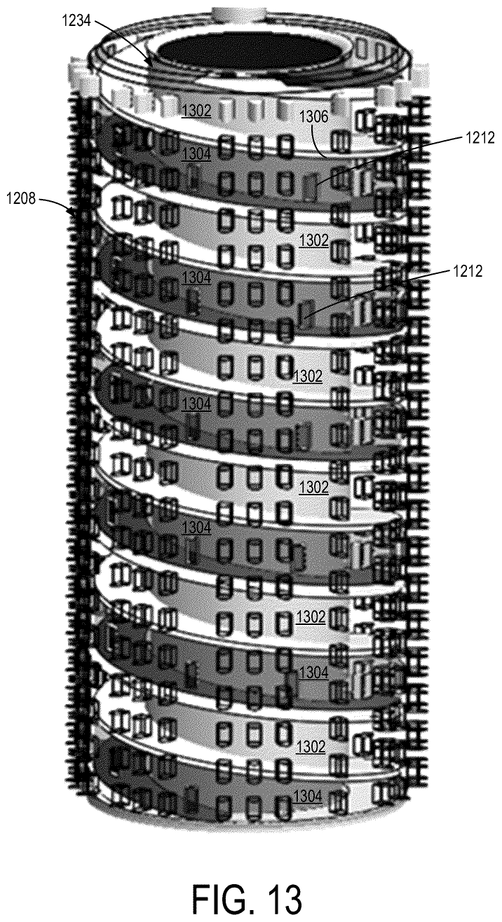

Turning now to FIG. 13, a partially cross-sectional view of the inflatable vacuum device 1110 according to the first example shown in FIG. 12 is shown, illustrating a positive pressure channel 1302 and a negative pressure channel 1304. The negative pressure channel 1304 may be in fluid communication with the first lumen connecting port 1204. The positive pressure channel 1302 may be in fluid communication with the second lumen port 1206. The positive pressure channel 1302 and the negative pressure channel 1304 may be separated by a separating element 1306. The separating element 1306 may be configured as a spiral. The separating element 1306 may be coupled to the outer wall 1232 (not shown in FIG. 13) and to the inner wall 1234. With respect to the separating element 1306, the negative pressure channel 1304 and positive pressure channel 1302 may be arranged at opposing sides. With the spiral configuration of the separating element 1306, the negative pressure channel 1304 and the positive pressure channel 1302 may also be configured as spiral shapes and may alternate, when viewed from a side perspective. The separating element 1306 may be configured to define a double spiral, wherein the positive pressure channel 1302 and the negative pressure channel 1304 are fluidly isolated from each other.

As noted above, the one or more openings 1212 may be included in the outer wall 1232 at positions corresponding to the negative pressure channel 1304. For example, a pattern of the one or more openings 1212 may follow the general spiral shape of the negative pressure channel 1304. The negative pressure channel 1304 may provide vacuum suction from the negative pressure source 110 to the target tissue and the positive pressure channel 1302 may provide outward pressure to the target tissue.

In some examples, the outer wall 1232 and inner wall 1234 may each be formed of two pieces. For example, a first spiral shaped piece may define the negative pressure channel and a second spiral shaped piece may define the positive pressure channel. The first and second spiral shaped pieces may be twisted together, coupling them together and forming the main body, with the outer wall and the inner wall being defined by the first and second spirals. When the inflatable vacuum device 1110 is formed as such by coupling two individual spirals, the two spirals may be formed of the same material or of different materials. For example, the inflatable vacuum device may be made of silicone, for example of thickness 0.5 mm. The separating element, when distinct from the channels, may be formed of the same material or a different material from the rest of the inflatable vacuum device.

The plurality of protrusions 1208, when included, may be configured to provide texture and external surface variation to the inflatable vacuum device 1110. A smooth surface element close to a vacuum port may adhere with a tight seal to the patient's mucosa, resulting in loss of blood and interstitial fluid flows, thereby resulting in tissue irritation, inflammation, and potentially death, as well as tissue tearing upon relative movements. The uneven or textured outer surface provided by the plurality of protrusions 1208 may thus reduce the potential for formation of a tight seal.

FIGS. 17A-17C show a second example of the inflatable vacuum device 1110. FIG. 17A shows a perspective view of the inflatable vacuum device 1110, FIG. 17B shows a lateral side view of the inflatable vacuum device 1110, and FIG. 17C shows a cross-sectional view of the inflatable vacuum device 1110 across cutting plane A-A′ shown in FIG. 17B. The inflatable vacuum device 1110 in the second example may include similar components to the first example described with respect to FIGS. 12 and 13. Shared components are given similar reference numbers and will not be reintroduced. Further, for the sake of brevity, aspects of the inflatable vacuum device 1110 that are similar in the second example compared to the first example will not be redescribed.

In the second example, a size, number, and/or arrangement of the protrusions 1208 may differ compared to the first example shown and described with respect to FIGS. 12 and 13. For example, each of the plurality of protrusions 1208 may be larger with respect to the body 1240 in the second example compared to the first example.

Further, the number of protrusions 1208 in the plurality of protrusions 1208 may be smaller in the second example compared to the first. For example, in the second example, each protrusion may correspond to an opening 1212, in a 1:1 ratio. In comparison, in the first example, the ratio of protrusions to openings may be 5:1, 10:1, 10:3, or similar.

Further still, in the second example, the protrusions 1208 may be arranged to follow the same spiral pattern as the plurality of openings 1212. Thus, with the 1:1 ratio of protrusions to openings and the arrangement of protrusions corresponding to the arrangement of the openings, the distance between openings and protrusions may be uniform throughout, thereby enabling adequate distance between suction from the opening and contact with the patient's mucosal tissue.

FIGS. 18A-18C show a third example of the inflatable vacuum device 1110 that does not include protrusions of the outer wall. FIG. 18A shows a perspective view of the inflatable vacuum device 1110, FIG. 18B shows a lateral side view of the inflatable vacuum device 1110, and FIG. 18C shows a cross-sectional view of the inflatable vacuum device 1110 across cutting plane A-A′ shown in FIG. 17B. The inflatable vacuum device 1110 in the third example may include similar components to the first and second examples described with respect to FIGS. 12-13 and 17A-C. Shared components are given similar reference numbers and will not be reintroduced. Further, for the sake of brevity, aspects of the inflatable vacuum device 1110 that are similar in the third example compared to the first and second examples will not be redescribed.

In the third example of the inflatable vacuum device 1110, the plurality of protrusions may not be present and the uneven outer surface of the inflatable vacuum device 1110 may be achieved via the differing pressures of the positive and negative pressure channels. For example, the inflatable vacuum device 1110 may be formed of one or more inflatable materials. The inflatable vacuum device may be deflated when initially inserted into the patient, and then inflated once in a desired position. The positive pressure channel may inflate corresponding sections of the outer wall more than sections corresponding to the negative pressure channel. Thus, the thickness 1242 of the body 1240 may vary, which may mitigate a tight seal that could result in tissue damage as described above.

Additionally, the second and third examples of the inflatable vacuum device 1110 shown in FIGS. 17A-18C include separated lumen ports. For example, the first lumen connecting port 1204 may be separated from the second lumen connecting port 1206, whereby the first lumen connecting port 1204 is arranged at a first side 1790 and the second lumen connecting port 1206 is arranged at a second side 1792 that is opposite the first side 1790. In such examples, the double lumen tube 1108 may split back into the first lumen 1104 and the second lumen 1106 at a second juncture positioned closer to the distal end 190 than the juncture 1102. In this way, the first lumen 1104 may couple to the first lumen connecting port 1204 and the second lumen 1106 may couple to the second lumen connecting port 1206 when positioned at the opposing sides of the inflatable vacuum device 1110.

In any of the first, second, and third examples of the third embodiment described with respect to FIGS. 11-13 and 17A-18C, the inflatable vacuum device may have an inflated diameter 1244 ranging between 5 and 10 mm. Inflation pressure may be adjusted based on tissue condition. For example, less positive pressure resulting in softer or more flexible walls may be utilized for healing sites and more positive pressure resulting in stiffer or more rigid walls may be utilized for stricture sites. Further still, the inflatable vacuum device herein described may be deflated, reinflated, inflated to different pressures, and/or replaced as a healing process progresses.

The fabrication of the dual lumen inflatable device can be as simple as the dual lumen tubing wound into a spiral within an external annular casing with only the negative pressure line having open ports. The positive pressure lumen would be sealed at the distal end. The outer thin soft surface of the external annular casing can be cast using PDMS (Polydimethylsiloxane) on a mold with a pattern of ports and pillars while the inner, top, and bottom surfaces are smooth and not porous. Although the same dual lumen tubing could be used for the entire system, having separate dual lumen tubing connecting the inflatable vacuum device may allow for different material and mechanical properties of the straight tubing and the spiraled tubing in the inflatable vacuum device. Alternatively, the inflatable vacuum device may be directly 3D printed using soft materials, such as using the Stratasys J750 DA (Digital Anatomy) 3D printer with Agilus30 Clear photopolymer material having rubber-like Shore 30A durometer value. The dual lumen tubing may be connected to this device using commercial dual lumen tubes at medical grade. The advantage of using 3D printing is that a patient's anatomy can be used to size the inflatable vacuum device. The Computed Tomography (CT) scan of the patient used prior to the surgery can be used to determine length and diameters of the device. Further, in some examples the severity of the tissue damage can provide guidance on the selection of the material properties used in the 3D printing.

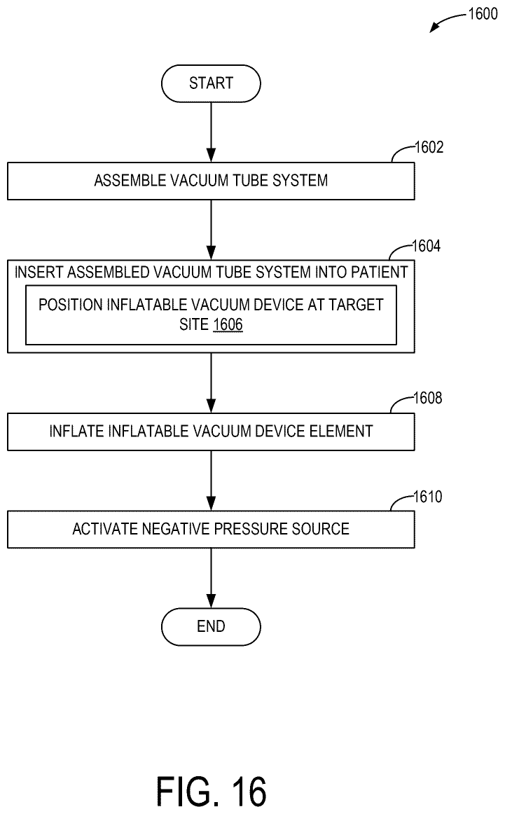

FIG. 16 shows a flowchart illustrating a method 1600 for the EVT system 100 according to the third embodiment of the present disclosure. The method 1600 is herein described with reference to the components of the system 100 with regard to the third embodiment of the present disclosure, wherein the system comprises a negative pressure source (e.g., negative pressure source 110), a positive pressure source (e.g., positive pressure source 112), and a tube system (e.g., vacuum tube system 102) comprising a double lumen tube (e.g., double lumen tube 1108) and a fluid collection element (e.g., inflatable vacuum device 1110), such as described with respect to FIGS. 11-13.

At 1602, method 1600 includes assembling vacuum tube system. As herein described with respect to the third embodiment of the present disclosure, the vacuum tube system may comprise the double lumen tube, including a positive pressure lumen and a negative pressure lumen. Assembling the EVT system may comprise connecting the positive pressure lumen to the positive pressure source and the negative pressure lumen to the negative pressure source.

Assembling the vacuum tube system may additionally comprise coupling the double lumen tube to the inflatable vacuum device. As described above, the inflatable vacuum device may comprise one or more lumen connecting ports (e.g., first lumen connecting port 1204 and second lumen connecting port 1206) that are configured to couple to the double lumen tube. For example, a negative pressure lumen connecting port may be configured to fluidly couple to a negative pressure lumen of the double lumen tube and a positive pressure lumen connecting port may be configured to fluidly couple to a positive pressure lumen of the double lumen tube.

When the vacuum tube system is assembled, the inflatable vacuum device may be in a first, deflated state.

At 1604, method 1600 includes inserting the assembled vacuum tube system (e.g., the inflatable vacuum device and double lumen tube) into a patient. The vacuum tube system herein described may be configured to be inserted via various approaches, such as transnasally, transorally, transrectally, percutaneously, or the like. For example, the vacuum tube system may be inserted transnasally, transorally, or percutaneously for upper gastrointestinal targets and transrectally or percutaneously for lower gastrointestinal targets. As a non-limiting example, the vacuum tube system may be inserted transrectally to reach a colonic anastomosis site, such as an end-to-end anastomosis created during a hemicolectomy procedure. During insertion, the deflated inflatable vacuum device may be positioned at a target site, as noted at 1606. For example, the inflatable vacuum device may be positioned to span the site, with a distal portion of the inflatable vacuum device being positioned at a distal side of the site and a proximal portion of the inflatable vacuum device being positioned at a proximal side of the site. Using the example of the end-to-end colonic anastomosis, the inflatable vacuum device may be positioned to center or approximately center at the anastomosis.

At 1608, method 1600 includes inflating the inflatable vacuum device. Inflating the inflatable vacuum device may comprise activating the positive pressure source. The inflatable vacuum device, as herein described, may include a negative pressure channel in fluid communication with the negative pressure source via the negative pressure lumen and a positive pressure channel in fluid communication the positive pressure source via the positive pressure lumen. Inflating the inflatable vacuum device may comprise activating the positive pressure to inflate the positive pressure channel. The negative pressure channel may be pre-formed and may not change configuration upon activation of the positive pressure source. In some examples, the inflatable vacuum device may be expanded after being positioned at a target site.

For example, if placed in the lower esophagus at the junction with the stomach, the most distal spiral of the positive pressure tube may be more expandable than the upper section where the anastomosis surgery occurred. Upon pressurizing the positive pressure line, the lower coil may expand greater within the upper stomach, which helps to anchor the device. Alternatively, at the lower esophageal sphincter, the pressures to constrict the esophagus may be greater so the positive pressure tubing may be thicker walled and stronger. By 3D printing from the CT scan, these modifications can be custom made specifically for the patient before the anastomosis surgery.

In other examples, the inflatable vacuum device may be expanded after insertion but prior to being positioned at a target set, the expanded inflatable vacuum device then being navigated to the target site. Once expanded, the positive pressure source may provide outward pressure to the target site. As an example, the inflatable vacuum device may be positioned within a lumen of the gastrointestinal tract (e.g., within a lumen of the colon or esophagus), and the positive/outward pressure may maintain a diameter of the lumen of the gastrointestinal tract to at least an outer diameter of the expanded inflatable vacuum device.

At 1610, method 1600 includes activating the negative pressure source to provide vacuum suction. As described above, inflatable vacuum device may be expanded after being inserted. The inflatable vacuum device, as described above, may comprise a plurality of openings corresponding to the negative pressure channel. The negative pressure source may create vacuum suction, which is distributed through the inflatable vacuum device via the plurality of openings. The inflatable vacuum device may allow for an even application of the suction across the entire wound surface. With the negative pressure source activated, the vacuum suction may pull wound exudate, blood, infectious material, and other fluid secretions through the negative pressure channel of the inflatable vacuum device and through the negative pressure lumen of the double lumen tube, to be collected in a secretion container of or coupled to the negative pressure source.

In some examples, one or more components of the EVT system according to the third embodiment may be used dynamically throughout the healing process. For example, early in the healing process the inflammation may be at its highest pressures of constriction while the fluid leakage is also at maximum rates. Therefore, the positive and negative pressures may be at their maximum values in the first few days after the anastomosis surgery, but then gradually reduced to match the needs of the tissue healing process. In another example, if the patient wishes to eat food, the stent may be adjusted to allow for involuntary peristalsis while the inflatable vacuum device stays in place. This is unlike mechanical stents that are permanently placed at one size and functional setting and allows the inflatable vacuum device herein described to be patient specific, both from an anatomy standpoint and from a wound type and stage standpoint.

In this way, the EVT system according to the third embodiment herein disclosed may provide both positive pressure and vacuum suction to a target site. As an example, the positive pressure may reduce potential of anastomotic stricture at an anastomosis site while the vacuum suction simultaneously provides vacuum suction to the anastomosis, removing wound exudate, blood, infectious material, and other fluid secretions and thus promoting healing.

The endoscopic vacuum therapy systems herein disclosed herein thus provide an efficient and easily accessible device for endoscopic vacuum therapy. As described above, currently, devices for endoscopic vacuum therapy are improvised by clinicians, which takes time and is not standardized. The endoscopic vacuum therapy system herein disclosed mitigates these inefficiencies of improvised devices by reducing the preparation and deployment time. The second embodiment herein disclosed provides an attachment system to quickly attach a foam sponge to the tube, thereby allowing larger diameter sponges to be used while still using a transnasal insertion approach. The third embodiment herein disclosed provides an inflatable inflatable vacuum device stent based system that incorporates both negative and positive pressure, thus providing a system that can both vacuum out undesirable fluids and debris to promote healing as well as put outward pressure on the mucosa to reduce potential strictures at the site.

The disclosure also provides support for an endoscopic vacuum therapy system, comprising: a negative pressure source, a tube comprising a plurality of openings at a distal end, wherein the tube is fluidly coupled to the negative pressure source at a proximal end, and a fluid collection element coupled to the tube, wherein the fluid collection element is configured to be compressed via a segmented dissolvable casing. In a first example of the system, the fluid collection element is a sponge. In a second example of the system, optionally including the first example, the segmented dissolvable casing comprises a plurality of gel shells arranged circumferentially around the foam sponge when the foam sponge is compressed, wherein the plurality of gel shells are spaced apart from one another. In a third example of the system, optionally including one or both of the first and second examples, the plurality of gel shells are each between 5 and 10 mm and are spaced apart from one another by gaps that are between 0.5 and 3 mm. In a fourth example of the system, optionally including one or more or each of the first through third examples the endoscopic vacuum therapy system further comprising an attachment system, wherein the attachment system comprises: an attachment clip arranged within a hollow interior of the foam sponge, the attachment clip comprising a hook configured to attach to one of the plurality of openings, and an attachment sheath. In a fifth example of the system, optionally including one or more or each of the first through fourth examples, the attachment clip comprises a hollow cylindrical body and one or more flanges coupled to the hollow cylindrical body and wherein the attachment sheath is configured to slide within a hollow interior of the hollow cylindrical body, the attachment sheath comprising a notch configured to align with the hook. In a sixth example of the system, optionally including one or more or each of the first through fifth examples, the fluid collection element comprises a main body, configured as a cylinder, and a conical tip, wherein the conical tip is arranged further towards the proximal end of the tube compared to the main body and the main body is positioned over the plurality of openings. In a seventh example of the system, optionally including one or more or each of the first through sixth examples, the conical tip of the fluid collection element is coupled to the tube via an adhesive, and further wherein the main body is not coupled to the tube via the adhesive.

The disclosure also provides support for a method for an endoscopic vacuum therapy (EVT) system, the method comprising: assembling a vacuum tube system by affixing a sponge to a distal portion of a tube, wherein the tube is connected to a negative pressure source, compressing the sponge with a segmented dissolvable casing, inserting the vacuum tube system into a patient, positioning the compressed sponge at a target site, and in response to the segmented dissolvable casing dissolving, activating the negative pressure source to provide vacuum suction through the sponge at the target site. In a first example of the method, the tube comprises a plurality of fenestrations and the sponge is affixed to the tube to cover the plurality of fenestrations. In a second example of the method, optionally including the first example, the sponge comprises an attachment clip, and affixing the sponge to the distal portion of the tube comprises: arranging an attachment sheath within the sponge and attachment clip, inserting the tube into the sponge through the attachment sheath, inserting a hook of the attachment clip into one of the plurality of fenestrations, and removing the attachment sheath. In a third example of the method, optionally including one or both of the first and second examples, the sponge comprises a cylindrically shaped main body and a conical tip. In a fourth example of the method, optionally including one or more or each of the first through third examples, affixing the sponge to the tube comprises adhering the conical tip to the tube proximal to the plurality of fenestrations.

The disclosure also provides support for a system, comprising: a negative pressure source, a positive pressure source, a double lumen tube comprising a negative pressure lumen and a positive pressure lumen, wherein the negative pressure lumen is coupled to the negative pressure source and the positive pressure lumen is coupled to the positive pressure source, and an inflatable vacuum device comprising a positive pressure channel and a negative pressure channel, wherein the positive pressure channel is in fluid communication with the positive pressure lumen and the negative pressure channel is in fluid communication with the negative pressure lumen. In a first example of the system, the inflatable vacuum device is configured as a hollow cylinder, wherein a main body of the hollow cylinder comprises the positive and negative pressure channels. In a second example of the system, optionally including the first example, the inflatable vacuum device further comprises a plurality of fenestrations, the plurality of fenestrations being positioned as openings into the negative pressure channel. In a third example of the system, optionally including one or both of the first and second examples, the negative pressure channel and the positive pressure channel are arranged in a double spiral configuration and are fluidly isolated from each other. In a fourth example of the system, optionally including one or more or each of the first through third examples, the inflatable vacuum device further comprises a plurality of protrusions that protrude away from an external wall of the inflatable vacuum device. In a fifth example of the system, optionally including one or more or each of the first through fourth examples, the inflatable vacuum device is configured to be inserted into a patient in a deflated state and inflated once positioned at a target site within the patient. In a sixth example of the system, optionally including one or more or each of the first through fifth examples, the negative pressure channel and the positive pressure channel are separate components configured to twist together to form the inflatable vacuum device.

At least FIGS. 2-3, 6, 8A-10, 12-13, and 17A-18C may be drawn to scale or approximately to scale.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. An endoscopic vacuum therapy system, comprising:
a negative pressure source;
a tube comprising a plurality of openings at a distal end, wherein the tube is fluidly coupled to the negative pressure source at a proximal end; and
a fluid collection element coupled to the tube, wherein the fluid collection element is configured to be compressed via a segmented dissolvable casing.

2. The endoscopic vacuum therapy system of claim 1, wherein the fluid collection element is a sponge.

3. The endoscopic vacuum therapy system of claim 2, wherein the segmented dissolvable casing comprises a plurality of gel shells arranged circumferentially around the foam sponge when the foam sponge is compressed, wherein the plurality of gel shells are spaced apart from one another.

4. The endoscopic vacuum therapy system of claim 3, wherein the plurality of gel shells are each between 5 and 10 mm and are spaced apart from one another by gaps that are between 0.5 and 3 mm.

5. The endoscopic vacuum therapy system of claim 2, the endoscopic vacuum therapy system further comprising an attachment system, wherein the attachment system comprises:
an attachment clip arranged within a hollow interior of the foam sponge, the attachment clip comprising a hook configured to attach to one of the plurality of openings; and
an attachment sheath.

6. The endoscopic vacuum therapy system of claim 5, wherein the attachment clip comprises a hollow cylindrical body and one or more flanges coupled to the hollow cylindrical body and wherein the attachment sheath is configured to slide within a hollow interior of the hollow cylindrical body, the attachment sheath comprising a notch configured to align with the hook.

7. The endoscopic vacuum therapy system of claim 1, wherein the fluid collection element comprises a main body, configured as a cylinder, and a conical tip, wherein the conical tip is arranged further towards the proximal end of the tube compared to the main body and the main body is positioned over the plurality of openings.

8. The endoscopic vacuum therapy system of claim 7, wherein the conical tip of the fluid collection element is coupled to the tube via an adhesive, and further wherein the main body is not coupled to the tube via the adhesive.

9. A method for an endoscopic vacuum therapy (EVT) system, the method comprising:
assembling a vacuum tube system by affixing a sponge to a distal portion of a tube, wherein the tube is connected to a negative pressure source;
compressing the sponge with a segmented dissolvable casing;
inserting the vacuum tube system into a patient;
positioning the compressed sponge at a target site; and
in response to the segmented dissolvable casing dissolving, activating the negative pressure source to provide vacuum suction through the sponge at the target site.

10. The method of claim 9, wherein the tube comprises a plurality of fenestrations and the sponge is affixed to the tube to cover the plurality of fenestrations.

11. The method of claim 10, wherein the sponge comprises an attachment clip, and affixing the sponge to the distal portion of the tube comprises:
arranging an attachment sheath within the sponge and attachment clip;
inserting the tube into the sponge through the attachment sheath;
inserting a hook of the attachment clip into one of the plurality of fenestrations; and
removing the attachment sheath.

12. The method of claim 10, wherein the sponge comprises a cylindrically shaped main body and a conical tip.

13. The method of claim 12, wherein affixing the sponge to the tube comprises adhering the conical tip to the tube proximal to the plurality of fenestrations.

14. A system, comprising:
a negative pressure source;
a positive pressure source;
a double lumen tube comprising a negative pressure lumen and a positive pressure lumen, wherein the negative pressure lumen is coupled to the negative pressure source and the positive pressure lumen is coupled to the positive pressure source; and
an inflatable vacuum device comprising a positive pressure channel and a negative pressure channel, wherein the positive pressure channel is in fluid communication with the positive pressure lumen and the negative pressure channel is in fluid communication with the negative pressure lumen.

15. The system of claim 14, wherein the inflatable vacuum device is configured as a hollow cylinder, wherein a main body of the hollow cylinder comprises the positive and negative pressure channels.

16. The system of claim 14, wherein the inflatable vacuum device further comprises a plurality of fenestrations, the plurality of fenestrations being positioned as openings into the negative pressure channel.

17. The system of claim 14, wherein the negative pressure channel and the positive pressure channel are arranged in a double spiral configuration and are fluidly isolated from each other.

18. The system of claim 14, wherein the inflatable vacuum device further comprises a plurality of protrusions that protrude away from an external wall of the inflatable vacuum device.

19. The system of claim 14, wherein the inflatable vacuum device is configured to be inserted into a patient in a deflated state and inflated once positioned at a target site within the patient.

20. The system of claim 14, wherein the negative pressure channel and the positive pressure channel are separate components configured to twist together to form the inflatable vacuum device.
